(19) United States
(12) Patent Application Publication
(10) Pub. No.: US 2021/0052781 A1
(43) Pub. Date: Feb. 25, 2021

(54) Transfer Ring

(71) Applicant: Micell Technologies, Inc., Durham, NC (US)

(72) Inventors: David W. Gaddy, Portsmouth, NH (US); Meagan M. Fagan, Raleigh, NC (US); James B. McClain, Ocracoke, NC (US)

(73) Assignee: Micell Technologies, Inc., Durham, NC (US)

(21) Appl. No.: 16/962,007

(22) PCT Filed: Jan. 16, 2019
(86) PCT No.: PCT/US2019/013802

§ 371 Date: Jul. 14, 2020

Related U.S. Application Data
(60) Provisional application No. 62/618,180, filed on Jan. 17, 2018.

(51) Int. Cl. A61L 29/08; B05B 13/02

(52) U.S. Cl. CPC A61L 29/08; B05D 1/04; B05B 13/0207

(57) ABSTRACT

Transfer rings for use in coating substrates and methods of utilizing same are disclosed. The transfer rings disclosed herein are useful in efficiently moving a plurality of substrates between different process chambers. For instance, the transfer rings are designed to collectively move a plurality of stents between coating and sintering chambers.

### CROSS-REFERENCE TO RELATED APPLICATIONS

The present application claims the benefit of U.S. Application Ser. No. 62/618,180, filed Jan. 17, 2018, entitled Transfer Ring, the disclosure of which is hereby incorporated herein by reference.

### BACKGROUND OF THE INVENTION

The present invention relates to apparatus used for coating substrates, was well as processes associated with same. The present application has particular applicability to apparatus and methodology associated with the coating of stents, although other substrates (medical or otherwise) can be coated utilizing the apparatus and methodologies disclosed herein.

Medical devices, such as implantable stents, drug-coated balloon catheters, or biomedical drug-delivery implants, are often coated with a combination of polymer and drug to create a coated construct that releases a therapeutic agent locally or systemically over time and/or provides a therapeutic effect local to the implanted device to enable reduced medical complications. For instance, coronary stents are often coated with a bioabsorbable polymer and anti-inflammatory or anti-proliferative drug such as, sirolimus (also referred to as rapamycin) or other therapeutic compounds of the limus class of drugs. After implantation the coating slowly degrades away, thereby releasing the drug into the patient and acting to enable healing of any tissues impacted by the introduction of the underlying stent structure. U.S. Pat. No. 8,298,565 (“the '565 Patent”), the disclosure of which is hereby incorporated by reference herein, discloses coated stents and methods for creating same. Although the '565 Patent will be referred to herein as teaching certain methodologies for coating stents or the like, other coating methodologies are also well known in the art and can benefit from the present invention.

A stent coating process similar to that disclosed in the '565 Patent is currently practiced by Miceli Technologies, Inc. of Durham, North Carolina. In short, that process typically involves the creation of a polymer and drug coating on a plurality of stents at a time through the following steps: (1) depositing a first polymer layer, (2) sintering that polymer layer (at an elevated temperature), (3) depositing a drug layer, (4) depositing a double polymer layer, (5) sintering that double polymer layer, (6) depositing a second drug layer, (7) depositing a triple polymer layer and (8) sintering the triple polymer layer. The polymer and drug depositing steps take place in polymer and drug coating chambers, respectively, while the sintering steps take place in a sintering chamber. Thus, the plurality of stents must be transferred multiple times among at least three different chambers to complete the coating process. Not only is individually moving these devices a time consuming process, but their relative diminutive size also makes the process all the more difficult.

Thus, there exists a need for a simple and efficient way to move a plurality of elements being coated among different coating and sintering chambers.

### BRIEF SUMMARY OF THE INVENTION

In general, the present invention provides an easy and efficient way of transferring stents or other medical devices among a plurality of coating and sintering chambers. Where it is well known to individually move such devices during the different processes, the present invention provides a bulk method of doing so, while also maintaining the overall quality of the coating process.

A first aspect of the present invention is a method of coating a plurality of stents. The method includes the steps of mounting the stents to a transfer ring to create a construct, placing the construct within a first coating chamber, coating the stents with a first material, removing the construct from the first coating chamber, placing the construct within a sintering chamber and sintering the coating previously deposited on the stents. In this method, the transfer ring does not negatively impact the sintering step.

Other embodiments of this first aspect may include the further steps of placing the construct in a second coating chamber and coating the stents with a second material. The placing steps may be performed by a robot or a machine driven arm. The steps of removing the coated stents from the transfer ring and rotating the transfer ring during the coating steps are also contemplated. With regard to the latter, the first and second coating chambers may impart a rotational force on the transfer ring. The mounting step may include mounting the stents on holders to the transfer ring and may include mounting 2-64 stents to the transfer ring. In certain embodiments, the first material may be a polymer and the second material may be a drug in crystalline form. The methodology may also further include repeating the placing, coating and sintering steps to create a multilayer coating on the stents. The coating may include three layers of polymer and two layers of drug. Sintering of the coating may be performed after depositing the polymer. In certain embodiments, the polymer is PLGA and the drug is sirolimus.

A further aspect of the present invention is a stent coating apparatus including a transfer ring, a first coating chamber, a second coating chamber and a sintering chamber. The transfer ring may be adapted to hold a plurality of stents and removably associated with the first and second coating chambers and the sintering chamber. In another embodiment, a separate sintering chamber may also be provided for the final sintering of the stents.

In other embodiments according to this second aspect, the transfer ring may be rotatably coupled with the first and second coating chambers. The transfer ring may be round and may include holders for holding the stents. In a preferred embodiment, the transfer ring includes 2-64 holders. The transfer ring may also include a plurality of standoffs that engage apertures in the first and second coating chambers and the sintering chamber. In particular, the transfer ring may include 12 standoffs and the first and second coating chambers and sintering chamber may each include 12 apertures for receiving the 12 standoffs. The transfer ring preferably provides an electrical connection between the first and second coating chambers and the stents. The apparatus may further comprise a robot or a machine driven arm for moving the transfer ring among the chambers.

### BRIEF DESCRIPTION OF THE DRAWINGS

The foregoing summary, as well as the following detailed description of the preferred embodiments of the invention will be better understood when read in conjunction with the appended drawings. For the purpose of illustrating the invention, they are shown in the drawings embodiments which are presently preferred. It should be understood, however, that the invention is not limited to the precise arrangements and instrumentality shown.

Referring to the figures, wherein like reference numbers represent like parts throughout the several views:

FIG. 1 is a perspective partial cutaway of a polymer coating chamber according to an embodiment of the present invention.

FIG. 2 is a perspective partial cutaway exploded view of a sintering chamber according to an embodiment of the present invention.

FIG. 3 is a perspective partial cutaway of a drug coating chamber according to an embodiment of the present invention.

FIG. 4 is a perspective partial cutaway of a transfer ring just prior to engagement with a chamber carousel according to an embodiment of the present invention.

FIG. 5 is a perspective exploded view of the transfer ring of FIG. 4.

### DETAILED DESCRIPTION

Reference will now be made in detail to the preferred embodiments of the present invention illustrated in the accompanying drawings. Wherever possible, the same or like reference numerals will be used throughout the drawings to refer to the same or like features. It should be noted that the drawings are in simplified form and are not drawn to precise scale. In reference to the disclosure herein, solely for purposes of convenience and clarity, directional terms such as top, button, above, below, and diagonal, are used with respect to the accompanying drawings. Such directional terms used in conjunction description of the drawings should not be construed to limit the scope of the invention in any manner not explicitly set forth. Additionally, the term “a” as used in the specification, means “at least one.” The terminology includes the words above specifically mentioned, derivatives thereof, and words of similar import.

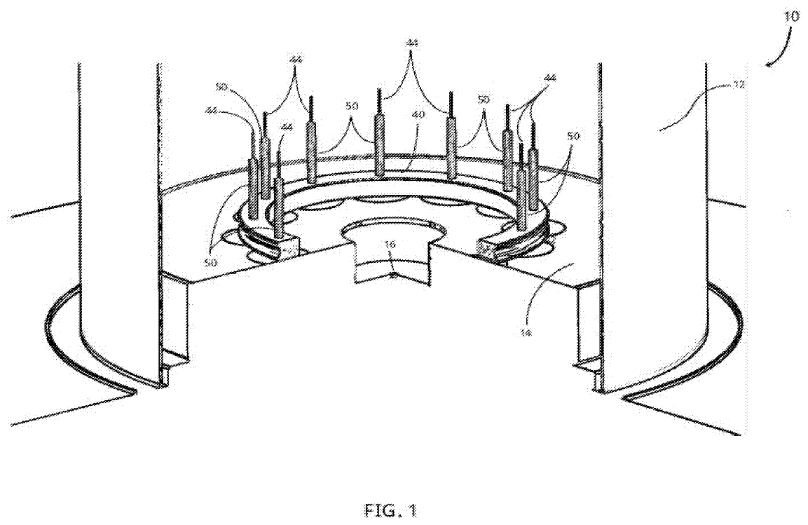

Referring to FIG. 1, a polymer coating chamber 10 according to the present invention is disclosed. This chamber is utilized in depositing single or multiple polymer layers on a stent during a coating process, like that taught in the '565 Patent. However, any type of known or hereinafter developed polymer coating chamber can be utilized. For instance, any of the chambers disclosed in U.S. patent application Ser. No. 11/877,591 (“the '591 Application”), the disclosure of which is hereby incorporated by reference herein, can be utilized in connection with the present invention. Although other chambers are contemplated, the present disclosure will focus on chamber 10 below as an example.

As shown, polymer coating chamber 10 includes an enclosure 12 and a carousel 14, which is rotatable with respect to other portions of the chamber. Chamber 10 also includes a nozzle 16 that allows for the introduction of polymer material into the chamber. Nozzle 16 is preferably attached to a polymer powder source that is capable of providing the powder through the nozzle and into enclosure 12. A heater block or the like may also be included to for heating any gas within nozzle 16 to overcome Joule-Thomson cooling. Any devices carried by carousel 14 are preferably covered by the injected powder, and the rotation of the carousel acts to ensure a uniform coverage. Although shown as a partial cut-out in FIG. 1, it is to be understood that carousel 14 is completely enclosed by enclosure, save for the opening provided by nozzle 16.

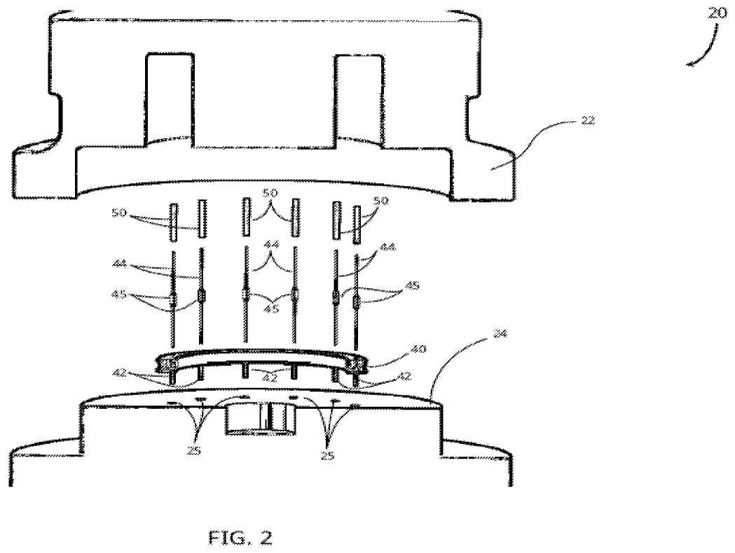

FIG. 2 shows a sintering chamber 20 that similarly includes an enclosure 22 and a carousel 24. The latter is similar in structure to carousel 14, but not rotatable, as rotation is not required during the sintering process. Chamber 20 also includes a heating source (not shown) that allows for the temperature within enclosure 22 to become elevated with respect to the ambient air so as to be capable of sintering the coating. For instance, in accordance with the present invention, chamber 20 is capable of subjecting devices placed therein to temperatures as high as 200 degrees Celsius. These temperatures are preferably suitable to sinter any polymer particles applied via above-discussed chamber 10. In practice, this typically involves subjecting the devices to approximately 2-10 minutes of these temperatures. In addition, chamber 20 is designed such that the interior of enclosure 22 can be pressurized up to 150-200 psi with a gas at conditions such that it is a non-solvent for the coating materials, so as to enhance the sintering process. Although a single sintering chamber 20 is shown, it is contemplated to employ a second sintering chamber (not shown) that can be utilized for a final sintering step. This chamber can apply a heat for a longer time period (e.g., 15-30 minutes) in order to provide a final sintering to the device resulting in more uniform coating.

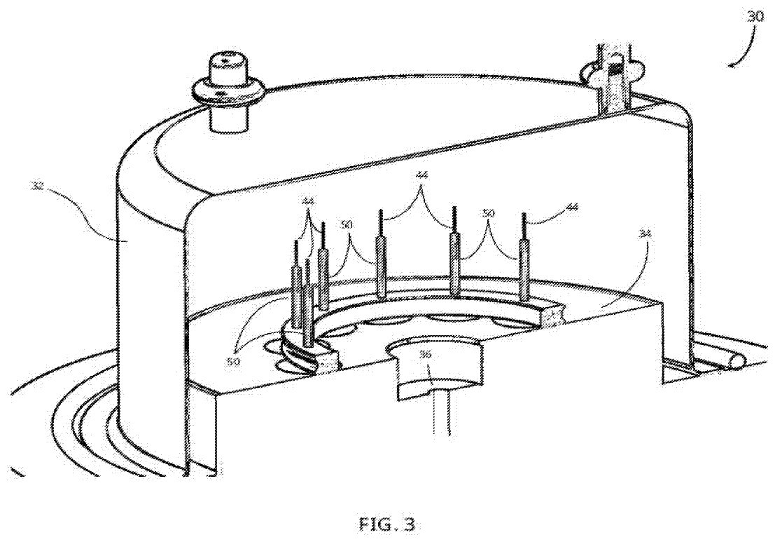

FIG. 3 shows a drug coating chamber 30, that like chambers 10 and 20, includes an enclosure 32 and a carousel 34. Again, carousel is similar in structure to carousels 14 and 24, for reasons that will be discussed below. Carousel 34 is also rotatable with respect to other aspects of chamber 30. Also like chamber 10, chamber 30 includes a nozzle 36. However, this nozzle is designed for the application of drug particles to the devices housed within enclosure 32. It is noted here, that these drug particles and/or the polymer particles coated in chamber 10 may be applied through the use of a supercritical fluid, as is discussed in the '565 Patent or via a dry powder spray process. Although shown as a partial cut-out in FIG. 3, it is to be understood that carousel 34 is completely enclosed by enclosure 32, save for the opening provided by nozzle 36.

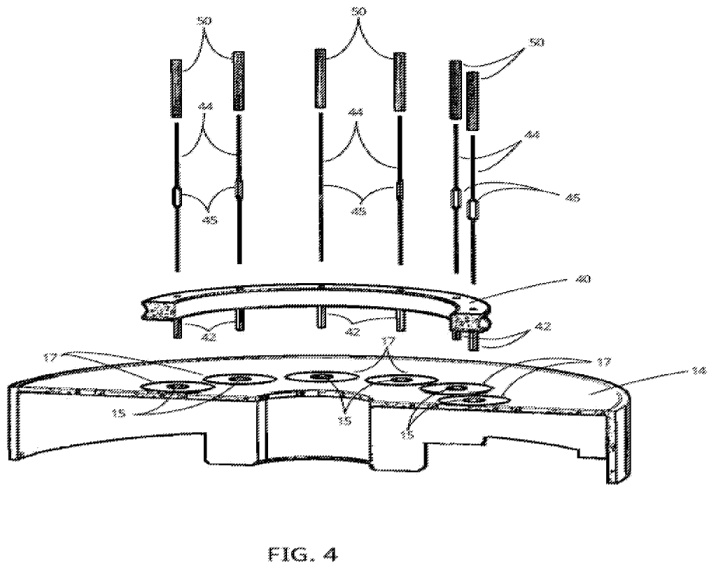

In each of above-discussed FIGS. 1-3, carousels 14, 24 and 34 are shown in cooperation with a common transfer ring 40. FIG. 4 details this cooperation in the exploded view showing the transfer ring and carousel 14. As shown, transfer ring 40 includes a plurality of standoffs 42 that are designed to be received within apertures 15 of carousel 14. It is to be understood that carousels 24 and 34 exhibit similar aperture patterns (not shown) so that transfer ring 40 can be universally utilized with the three different carousels. Although transfer ring 40 is depicted exhibiting a certain shape (e.g., circular) and size, it is noted that the device can be many different shapes and sizes. For instance, it is contemplated to construct the transfer ring in a polygonal shape or as a double-ringed structure. The only limitation is that the carousels with which the transfer rings are to be associated must exhibit a similar structure (e.g., square shaped aperture patterns).

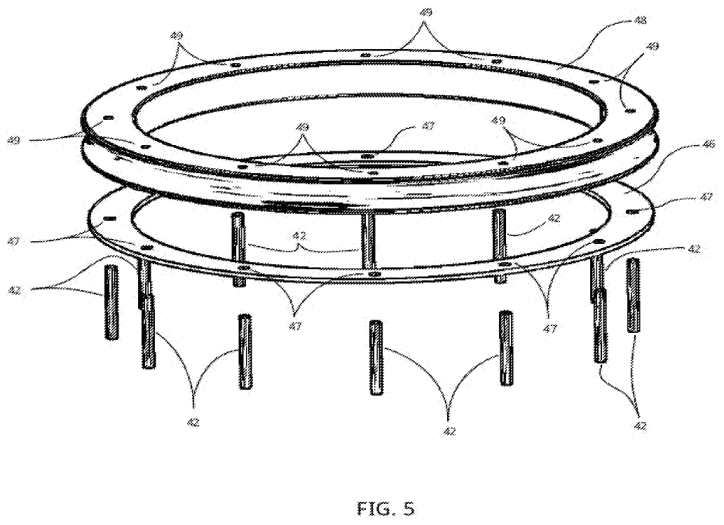

Transfer ring 40, as shown in FIGS. 1-4, also includes a plurality of stent holders 44 upon which are mounted a plurality of stents 50. It is noted that these stent holders can take any form, including the retaining designs disclosed in the '591 Application. For instance, as best shown in FIG. 4, each of holders 44 includes an enlarged section 45 that is designed to capture and hold in place stents 50. As will be discussed more fully with reference to FIG. 5, the design of transfer ring 40 is preferably such that an electrical charge can be provided to stents 50 in both chambers 10 and 30.

FIG. 5 depicts transfer ring 40 in an exploded manner (save for holders 44). As shown, in addition to the above-discussed standoffs 42 and holders 44, the ring includes a conductor ring 46 and an insulator ring 48. Standoffs 42 are designed to extend through holes 47 formed in conductor ring 46 and at least partially through holes 49 formed in insulator ring 48. This allows for the standoffs to hold the two rings together in the completed construct. In the design shown, standoffs 42 and conductor ring 46 are constructed of a metallic material (e.g., stainless steel), while insulator ring 48 is constructed of a polymer material, (e.g., PEEK or PTFE). This construction is such that it allows an electrostatic potential to be provided to the individual stents 50, while in coating chambers 10 and 30. In particular, carousels 14 and 34 include an electrical connection provided within apertures 15 that cooperates with standoffs 42. With particular reference to FIG. 4, a Teflon ring 17 or the like is provided around apertures 15 and extends into carousels 14 and 34. This ring insulates the remainder of the carousels preventing charge transfer from any charge provided by the electrical connection to the stents/stent holders. In fact, it is contemplated to provide the remainder of the structures of the chambers to be provided with an opposite charge. This electrostatic potential further acts to direct polymer and drug particles to the stents. Of course it is contemplated to construct transfer ring 40 and ring 17 of many different materials in accordance with the present invention. As shown in FIG. 2, carousel 24 includes similar apertures 25, although an electrical connection is not provided in chamber 20.

The construction of transfer ring 40 and its cooperation with chambers 10, 20 and 30 allows the plurality of stents 50 to be transferred among the chambers at the same time. In other words, the transfer rings (and hence the stents) can be lifted and moved from one chamber to another in a simple step. This significantly reduces the time and effort required in moving individual stents (more particularly stents mounted on stent holders) or other devices among the chambers. Moreover, the construction of transfer ring 40 is such that an electrostatic potential can be provided to the stents 50 individually in both the poly chamber 10 and drug chamber 30. As this allows for the stents to be oppositely charged from the polymer and drug particles, it can aid in gaining a more uniform coating of those particles on the stents, as well as significantly reduce waste during the coating processes. Moreover, the heat capacity properties (e.g., thermal mass) of transfer ring 40 are such that the ring doesn't negatively impact the sintering process. In this regard, the ring preferably exhibits a construct in which its overall heat capacity and transfer properties do not prevent even sintering of the portion of stents 50 that lie adjacent to insulator ring 48. This cures a problem that would be seen with the use of a device with greater thermal mass, such as a carousel, being moved between polymer or drug chambers and a sintering chamber. There, the larger construct would negatively impact the sintering of at least the portions of the stents lying closest to the carousel.

Although discussed as being comprised of both conductive and insulating materials, it is contemplated to form transfer ring 40 solely of one of those materials. Either type of material may allow for an electrostatic charge to ultimately be provided to stents 50. For instant, an electrostatic charge could be provided even when solely insulating materials are utilized. However, the use of both conductive and insulating materials allows for any charges to be focused in the areas of the stents, which may in turn allow for oppositely charged polymer and drug particles to be attracted to those areas. This may aid in reducing waste of those materials.

In use, transfer ring 40 is first loaded with a plurality of stents 50. This step is performed by sliding each stent 50 over a different holder 44 until it is captured by enlarged section 45. As shown, transfer ring 40 is capable of holding twelve stents 50. Of course, in other embodiments, the ring can be constructed to hold any number of stents or other devices.

The transfer ring 40 and stent 50 construct is then placed within polymer coating chamber 10. After an electrical connection is established and the stents are properly charged, oppositely charged polymer powder is provided via nozzle 16 into enclosure 12. It is noted here that in addition to the cooperation between chamber 10 and transfer ring 40 allowing for stent 50 to be properly charged, holders 44 aid in the proper charging. Indeed, the holders can be designed as in the '591 Application, which discusses the electrical connection between holders and stents.

After a layer of polymer particles is coated on the stents in chamber 10, the transfer ring 40 and stent 50 construct is moved to sintering chamber 20. Again, this can be accomplished in a single step, as opposed to the multiple steps required in the individual moving of stents. The entire construct is then subjected to elevated temperatures for enough time to allow for the sintering of the polymer particles into a continuous or semi-continuous film. As noted above, the construction of transfer ring 40 is such that this sintering can be done in an efficient manner without the need for extra sintering time that would be required if the ring negatively impacted the heat transfer to stents 50.

The transfer ring 40 and stent 50 construct can then be moved to drug coating chamber 30, which is utilized to inject drug particles. These drug particles can be any type of drug, but are preferable macrolide immunosuppressive drugs in crystalline form that may be combined with a super critical fluid, compressed gas or other fluid or propellant and then injected through nozzle 36. This allows for the crystallinity of the drug to be maintained throughout the coating process. Again, during this coating process, stents 50 may be charged in a manner in which the drug is attracted thereto.

Thereafter, the polymer coating, sintering and drug coating processes can be repeated until a suitable coating is provided on stents 50. The ease of removal and placement of the transfer ring 40 and stent 50 construct allows for easy transition between these steps. Thus, any number of processes may be performed without the placement of stents 50 being a significant limiting factor in the overall efficiency of the coating process.

It is to be understood that the present invention is not limited to the coatings of stents, and that the present invention may have applicability to the coatings of other devices. For instance, transfer ring 40 may be designed to accommodate other medical devices (e.g., balloons or implants) and allow for transfer among different chambers in the same or similar coating processes as those described above. It is also to be understood that the particular transfer ring 40 construct shown and described herein may vary in accordance with the present invention. Indeed, the transfer ring may be tailored to existing chambers or to a given device that is to be coated. Moreover, although shown as having the capability of holding 12 stents, it is noted that transfer rings in accordance with the present invention may hold any number of stents, for instance anywhere between 2-64. Finally, although discussed and shown as being a device suitable for movement by hand, it is to be understood that transfer ring 40 may be moved in an automated process involving robotic arms or the like. In particular, an articulatable, machine driven arm may engage and carry the transfer ring (and the devices associated therewith) among the different coating and sintering chambers. Thus, the present invention lends its self well to the overall automation of the coating processes discussed herein and in the incorporated patent and patent applications. In certain embodiments, it may be beneficial to move both the transfer ring and carousel among the different chambers via the robot or machine driven arm. If multiple transfer rings are provided, it is possible to semi-continuously or continuously utilize the chambers so that multiple groups of devices can be being coated at a given time. In this regard, it may be beneficial to employ more than one of each type of chamber. It is also contemplated to integrally form the transfer ring with the carousel in certain embodiments. Of course, this integral formation should be such that the benefits of the transfer ring are still seen in the final construct.

Certain patents and patent applications are discussed and incorporated by reference above. It is to be understood that these references disclose example coating methods and apparatus for use in such process. However, other methods and apparatus can be employed in conjunction with the present invention. For instance, U.S. patent application Ser. Nos. 12/298, 459; 12/426,198; 12/751,902; 12/762,007; 13/086,335; 13/445,723; 11/158,724; 11/877,591; 12/443,959; 12/522,379; 12/595,848; 12/601,101; 12/738,411; 12/504,597; 12/729,580; 13/384,216; 13/809,324; 14/131,878; 12/729,156; 12/729,603; 13/014,632; 13/090,525; 13/229,473; 13/340,472; 12/748,134; 14/122,862; 12/648,106; 14/238,677; 14/401,496; 14/402,839; 14/352,664; 14/437,097; 14/207,336; 14/278,367; 10/156,970 disclose other methods and apparatus for creating coatings. The disclosures of each of these applications are also incorporated by reference herein in their entireties. It is to be understood that the present invention may be utilized with the methods and apparatus disclosed in these further applications.

Although the foregoing description and appended drawings detail transfer rings in which stents or the like are mounted in a vertical fashion, it is contemplated to mount such devices in a horizontal or other fashion. For instance, it is envisioned that a transfer ring in accordance with the present invention may exhibit a ferris wheel-like configuration where the devices are mounted in a horizontal position on a rotating and electrostatically effective fixture. This transfer ring can then be moved among the different chambers. Likewise, although present application largely focuses on stents in general, it is noted that the present invention can be tailored for use with any type of substrate, medical or otherwise. For instance, it is contemplated to coat medical devices such as stents, balloons, stent-grafts, catheters, orthopedic implants, embolization coils, vascular filters and the like. Moreover, while stents are discussed generally herein, it is to be understood that such structures can include any type of stent, e.g., expandable, self-expanding and/or bioabsorbable stent devices. Of course, depending upon the type of substrate being coated, the transfer rings discussed herein may need to be modified to properly hold such devices and present them for proper coating.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of coating a plurality of medical devices comprising:
mounting the medical devices to a transfer ring to create a construct;
placing the construct within a first coating chamber;
coating the medical devices with a first material;
removing the construct from the first coating chamber;
placing the construct within a sintering chamber; and
sintering the medical devices,
wherein the transfer ring does not negatively impact the sintering step.

2. The method of claim 1, further comprising the steps of placing the construct in a second coating chamber and coating the medical devices with a second material.

3. The method of claim 2, wherein the placing steps are performed by a robot or a machine driven arm.

4. The method of claim 2, further comprising the step of removing the coated medical devices from the transfer ring.

5. The method of claim 4, further comprising the steps of rotating the transfer ring during the coating steps.

6. The method of claim 5, wherein the first and second coating chambers impart a rotational force on the transfer ring.

7. The method of claim 1, wherein the mounting step includes mounting the medical devices on holders to the transfer ring.

8. The method of claim 1, wherein the mounting step includes mounting 2-64 medical devices to the transfer ring.

9. The method of claim 2, wherein the first material is a polymer and the second material is a drug in crystalline form.

10. The method of claim 9, further comprising repeating the placing, coating and sintering steps to create a multilayer coating on the medical devices.

11. The method of claim 10, wherein the coating includes three layers of polymer and two layers of drug.

12. The method of claim 10, further comprising the steps of sintering the coating after depositing polymer.

13. The method of claim 9, wherein the polymer is PLGA and the drug is sirolimus.

14. The method of claim 1, wherein the medical devices are stents.

15. A medical device coating apparatus comprising:
a transfer ring;
a first coating chamber;
a second coating chamber; and
a sintering chamber,
wherein the transfer ring is adapted to hold a plurality of medical devices and is removably associated with the first and second coating chambers and the sintering chamber.

16. The apparatus of claim 15, wherein the transfer ring is rotatably coupled with the first and second coating chambers.

17. The apparatus of claim 15, wherein the transfer ring is round.

18. The apparatus of claim 15, wherein the medical devices are stents and the transfer ring includes holders for holding the stents.

19. The apparatus of claim 18, wherein the transfer ring includes 2-64 holders.

20. The apparatus of claim 15, wherein the transfer ring includes a plurality of standoffs that engage apertures in the first and second coating chambers and the sintering chamber.

21. The apparatus of claim 20, wherein the transfer ring includes 12 standoffs and the first and second coating chambers and sintering chamber each include 12 apertures for receiving the 12 standoffs.

22. The apparatus of claim 15, wherein the transfer ring provides an electrical connection between the first and second coating chambers and the medical devices.

23. The apparatus of claim 15, further comprising a robot or a machine driven arm for moving the transfer ring among the chambers.
